(19) United States
(12) Patent Application Publication
(10) Pub. No.: US 2012/0282190 A1
(43) Pub. Date: Nov. 8, 2012

(54) COMPOSITIONS, METHODS, AND KITS COMPRISING A DRY SHAMPOO COMPOSITION

(75) Inventor: James Donald Hammer, Uxbridge, MA (US)

(73) Assignee: Pharmosol Corporation, South Easton, MA (US)

(21) Appl. No.: 13/503,576

(22) PCT Filed: Oct. 27, 2010
(86) PCT No.: PCT/US10/54209

§ 371 Date: Jul. 31, 2012

Related U.S. Application Data
(60) Provisional application No. 61/255,281, filed on Oct. 27, 2009.

(51) Int. Cl. A61K 8/04; A61P 17/00; A61K 9/12; A61P 33/14; A61Q 5/02; A61Q 5/06

(52) U.S. Cl. 424/47; 510/120; 424/45

(57) ABSTRACT

The present invention relates to compositions, methods, and kits comprising a dry shampoo composition. In some cases, a dry shampoo composition is provided as an aerosol. A dry shampoo composition may comprise at least one clay material, at least one starch material, and at least one carrier material. The composition may additional comprise one or more additive components, such as an oil-absorbing compound, a suspending agent compound, and/or a perfume.

### FIELD OF INVENTION

The present invention generally relates to compositions, methods, and kits comprising a dry shampoo composition, and more particularly to a dry shampoo composition provided as an aerosol.

### BACKGROUND

Aerosol dry shampoo compositions are known (e.g., Oscar Blandi Dry Shampoo Spray, Ojon Rub-Out Dry Cleanser, Naturia Dry Shampoo by Rene Furterer) and generally work by spraying an aerosol comprising a dry shampoo composition onto hair of a subject. A carrier material in the dry shampoo composition evaporates and a powder remains. The powder may absorb sebum from the hair and the powder may then be removed, at least in part, by brushing the hair. By this means, the hair is cleaned. However, known aerosol dry shampoo compositions generally leave a colored residue on the hair.

### SUMMARY OF THE INVENTION

The present invention generally relates to compositions, methods, and kits comprising a dry shampoo composition, and in particular, to an aerosol dry shampoo composition. The subject matter of the present invention involves, in some embodiments, interrelated products, alternative solutions to a particular problem, and/or a plurality of different uses of one or more compositions and/or methods.

In some embodiments, the present invention is directed towards compositions. In a first embodiment, an aerosol dry shampoo composition comprises a carrier material in a weight percentage between about 1% and about 35%, a starch material in a weight percentage about 3% and about 5%, a clay material in a weight percentage about 2% and about 4%, and a propellant in a weight percentage about 75% and about 80%.

In another embodiment, an aerosol dry shampoo composition consists of or consists essentially of stearalkonium hectorite in a weight percentage between about 2% and about 4%, aluminum starch octenylsuccinate in a weight percentage between about 3% and about 5%, ethanol in a weight percentage between about 1% and about 20%, cyclomethicone in a weight percentage between about 0.1% and about 15%, silica in a weight percentage between about 0.1% and about 0.5%, and isobutane in a weight percentage between about 75% and about 80%.

In yet another embodiment, an aerosol dry shampoo composition consists or consists essentially of stearalkonium hectorite in a weight percentage between about 2% and about 4%, aluminum starch octenylsuccinate in a weight percentage between about 3% and about 5%, ethanol in a weight percentage between about 1% and about 20%, cyclomethicone in a weight percentage between about 0.1% and about 15%, silica in a weight percentage between about 0.1% and about 0.5%, isobutane in a weight percentage between about 75% and about 80%, and at least one additive in a weight percentage between about 0.1% and about 10%.

### DETAILED DESCRIPTION OF THE INVENTION

The present invention provides methods and compositions relating to a dry shampoo composition. In some embodiments, a dry shampoo composition may be essentially colorless and/or transparent following application to hair, and, in some embodiments, the dry shampoo composition may leave little or no colored residue on the hair. Such compositions may reduce or eliminate an oily and/or unaesthetic appearance of hair. In some aspects, a dry shampoo composition comprises at least one starch material, at least one clay material, and at least one carrier material. The dry shampoo composition may be provided as an aerosol (e.g., an aerosol dry shampoo composition).

The compositions of the invention may be used for topical application to hair (e.g., human hair), in particular, when the hair has become excessively greasy (e.g., due to an accumulation of sebum). The compositions, following application to hair, may be transparent or substantially transparent (e.g., leaving no color and/or flakes). This may be particularly important in instances where the dry shampoo composition is being applied to darker colored hair (e.g., brunette hair, black hair). Additionally, the compositions may not weigh-down the hair and/or may leave little or no residue. The composition may be used by a subject a plurality of times, wherein the hair of the subject is not washed (e.g., with soap and water) between uses.

In some embodiments, a dry shampoo composition is provided as an aerosol (e.g., an aerosol dry shampoo composition). The term “aerosol dry shampoo composition,” as used herein, refers to a composition comprising a dry shampoo composition and a propellant. When a dry shampoo composition is provided as an aerosol as compared to a powder, it may advantageously allow for the dry shampoo composition to be applied in a diffusive manner, and may increase the transparency of the dry shampoo composition when compared to powder application. Methods for forming an aerosol dry shampoo composition are described herein.

It should be understood, however, that a dry shampoo composition may be provided using a different delivery method such as applying by hand directly to the hair, using a container fitted with a pump to dispense a liquid composition (e.g., an atomizers), as will be known to those of ordinary skill in the art. In some embodiments, a dry shampoo composition may be provided in a pump aerosol container which utilizes compressed air as the propellant.

A dry shampoo composition may comprise a plurality of components including, but not limited to, clay materials, starch materials, compounds for oil absorption, carrier materials, and/or additive components. A dry shampoo composition may comprise more than one of each type of component, that is, at least one, at least two, at least three, at least four, at least five, or more, of each type of component. The number of each type of component may be the same or different (e.g., the composition may comprise one type of clay material, one type of starch material, two types of carrier materials, and one or more additive components). Possible compounds and materials that may be used for each type of component are described herein.

In some embodiments, a dry shampoo composition comprises at least one type of clay material. A clay material may aid in oil absorption and/or act as a suspending agent. A clay material may be modified, for example, to include organic moieties. Such a modification may increase the dispersion of the clay material in an alcohol as compared to a non-modified version the clay, may reduce or eliminate the static charge on the product, and/or may aid in the binding of the clay to hair. Those of ordinary skill in the art will be aware of methods and techniques for modifying a clay material, such as with organic groups. Non-limiting examples of classes of clay materials include bentonite, hectorite, and kaolin. Non-limiting examples of modified bentonites and hectorites include stearalkonium hectorite, stearalkonium bentonite, quaternium-18 bentonite, and quaternium-18 hectorite. In one embodiment, the clay material is stearalkonium hectorite. In embodiments where a dry shampoo composition is provided as an aerosol, the total percentage weight of the clay material may be between about 1% and about 10%, between about 1% and about 8%, between about 1% and about 5%, between about 2% and about 6%, between about 3% and about 5%, between about 1% and about 4%, between about 2% and about 4%, or the like, of the total weight of the aerosol dry shampoo composition (a combination of the dry shampoo composition and the propellant). In a particular embodiment, the clay material is present in an amount between about 2% and about 4%. In some cases, a higher percentage of clay material may be provided as compared to previously known dry shampoo compositions.

In some embodiments, a dry shampoo composition comprises at least one starch material. A starch material may function as an oil absorber. Non-limiting examples of starch materials include cornstarch, potato starch, tapioca starch, rice starch, wheat starch, and cassaya starch. A starch material may be modified or unmodified. A modified starch material is a starch which has been derivatized or altered by processes known to those of ordinary skill in the art, such as esterification, etherification, oxidation, acid hydrolysis, crosslinking, or enzyme conversion. Non-limiting examples of modified starch materials include aluminum starch octenylsuccinate, sodium starch octenylsuccinate, calcium starch octenylsuccinate, distarch phosphate, hydroxyethyl starch phosphate, hydroxypropyl starch phosphate, sodium carboxymethyl starch, and sodium starch glycolate. It should be understood, however, that in some embodiments, a starch material may be replaced by another oil-absorbing powder, for example, cellulose, chalk, talc, fullers earth, etc. In a particular embodiment, the starch material is aluminum starch octenylsuccinate. In embodiments where a dry shampoo composition is provided as an aerosol, the total percentage weight of the starch material may be between about 0.01% and about 8%, 1% and about 7%, between about 1% and about 5%, between about 1% and about 3%, between about 3% and about 7%, between about 3% and about 5%, or the like, of the total weight of the aerosol dry shampoo composition (a combination of the dry shampoo composition and the propellant). In a particular embodiment, the starch material may be present in an amount between about 3% and about 5%. Typically, a starch material is present in a weight percent less than about 8%. In some cases, a lower percentage of starch material may be provided as compared to previously known dry shampoo compositions. This may aid in substantially reducing and/or eliminating any amount of colored residue that may be caused by a starch material as compared to the previously known dry shampoo compositions.

In some embodiments, a dry shampoo composition comprises at least one carrier material. A carrier material typically is a volatile liquid. In some cases, a carrier material is a volatile alcohol including, but not limited to, ethanol or isopropanol. In some cases, a carrier material may be a volatile silicone compound. Volatile silicone compounds generally have an atmospheric pressure boiling point of less than about 220° C., or between about 50° C. and about 220° C., and contain between about 3 and about 7 silicon atoms. Non-limiting examples of volatile silicone compounds include polydimethylsiloxanes (e.g., having a viscosity less than about 5 cSt at 25° C.), cyclomethicone, cyclohexane siloxane, decamethyltetrasiloxane, octamethyltrisiloxane, decamethylpentasiloxane, decamethylcyclopentasiloxane, octamethylcyclotetrasiloxane, trimethylsilylamodimethicone, phenyl trimethicone, hexamethyidisiloxane, dimethylsiloxane/methylalkylsiloxane, or combinations thereof. In a particular embodiment, the volative silicone component is cyclomethicone. In embodiments where a dry shampoo composition is provided as an aerosol, the total percentage weight of the carrier material may be between about 0.1% and about 50%, between about 0.1% and about 40%, between about 1% and about 35%, between about 5% and about 50%, between about 10% and about 40%, between about 15% and about 40%, of the total weight of the aerosol dry shampoo composition (a combination of the dry shampoo composition and the propellant). In a particular embodiment, the carrier material is present in an amount between about 1% and about 35%. In some cases, the carrier material may comprise a mixture of an alcohol, such as ethanol or isopropanol, and a volatile silicone compound, such as cyclomethicone or cyclohexane siloxane. In a particular embodiment, the carrier material may comprise an alcohol (e.g., ethanol) in an amount between about 1% and about 20% and a volatile silicone compound (e.g., cyclomethicone) in an amount between about 0.1% and about 15%

In some embodiments, the dry shampoo composition may comprise at least one additive component (e.g., at least two, three, four, five, or more, additives). If more than one additive is comprised in the dry shampoo composition, the additives may or may not have the same function(s) or characteristics (e.g., in the case of two additives, one may function as a perfume and the other may be an anti-dandruff agent, or both may function as an anti-dandruff agent). Non-limiting examples of additive components include: antioxidants; essential oils; perfumes; waxes; fillers; hair-fixative polymers; deodorizing agents; pediculicides; anti-dandruff agents; cosmetic and/or dermatological active agents including emollients, moisturizers, vitamins (e.g., vitamin B complexes (e.g., including thiamine, nicotinic acid, biotin, pantothenic acid, choline, riboflavin, vitamin B6, vitamin B12, pyridoxine, inositol, carnitine), vitamins A, C, D, E, K and their derivatives (e.g., vitamin A palmitate) or pro-vitamins), essential fatty acids, sunscreens, herb and/or plant extracts (e.g., aloe); dispersing or suspending agents (e.g., silica); pharmaceutically active agents (e.g., poly(2-hydroxystearic acid); anti-static agents (e.g., tricetyl methyl ammonium chloride); pearlescent aids (e.g., such as coated mica, ethylene glycol distearate); opacifiers (e.g., tin); preserving agents (e.g., 1,2-dibromo-2,4-dicyano butane, benzyl alcohol, methyl paraben, propyl paraben, imidazolidinyl urea); coloring agents or dyes; odor neutralizers; and sequestering agents; or combinations thereof. In some embodiments, the additive component may be capable of absorbing oils and/or may aid as a suspending or dispersing agent. In other embodiments, the additive component may provide a cooling effect to the subject when the composition is applied (e.g., menthol). In a particular embodiment, an additive component is silica. The silica may function as an oil-absorbing compound and/or a suspending agent. Other suitable additive components will be known to those of ordinary skill in the art.

In some cases, an additive component may be a hair-fixative polymer. The term “hair-fixative polymer” is given its ordinary meaning in the art and generally refers to a film-forming polymer which is soluble in selected solvent(s) (e.g., present in the dry shampoo composition) and is capable of forming a film and/or holding the hair of a user in place after evaporation of the selected solvent(s). Non-limiting examples of hair fixative polymers include octylacrylamide/acrylates/butylaminoethyl/methacrylate copolymer, PVP, VP/VA copolymer, acrylates copolymer, polyurethane-14/AMP-acrylates copolymer, and combinations thereof. Other suitable hair-fixative polymers will be known to those of ordinary skill in the art.

In some cases, an additive component is a deodorizing agent. Non-limiting examples of deodorizing agents include cyclodextrins, zinc undecylenate, undecylenic acid, natural fragrance oils, and combinations thereof.

In some cases, an additive component is an anti-dandruff agent. Non-limiting examples of anti-dandruff agents include ketoconazole, selenium sulfide, coal tar, salicylic acid, zinc pyrithione, benzethonium chloride, benzalkonium chloride, chlorhexidine, chloramine T, chloramine B, sulphur (e.g., in various forms), cadmium sulphide, wood tar, and combinations thereof.

In some cases, an additive component is a pediculicide (e.g., synthetic or natural pediculicide). Non-limiting examples of pediculicides include pyrethrins, permethrin, piperonyl butoxide, and natural agents, such as aniseed oil, coconut oil, neem oil, tea tree oil, or combinations thereof.

In embodiments where a dry shampoo composition is provided as an aerosol, the total percentage weight of the at least one additive component may between about 0.01% and about 10%, between about 0.01% and about 5%, between about 0.1% and about 3%, between about 0.01% and about 1%, between about 0.1% and about 5%, between about 1% and about 5%, or the like, of the total weight of the aerosol dry shampoo composition (a combination of the dry shampoo composition and the propellant).

In some embodiments, the solid components (e.g. starch material, clay material, additive components) may be provided as a powder comprising a plurality of particles of the component. The particles may be polydisperse (e.g., having a range of different sizes), or in some cases, the particles may be monodisperse or substantially monodisperse, (e.g., having a homogenous distribution of diameters), such that no more than about 10%, about 5%, about 3%, about 1%, about 0.03%, or about 0.01% of the particles have an average diameter greater than about 10%, about 5%, about 3%, about 1%, about 0.03%, or about 0.01% of the average diameter. The “average diameter” of a plurality of particles, as used herein, is the arithmetic average of the diameters of the particles. Those of ordinary skill in the art will be able to determine the average diameter of a plurality of particles, for example, using laser light scattering or other known techniques. As non-limiting examples, the average diameter of the plurality of particles of the starch component may be about 100 microns, about 50 microns, about 30 microns, about 25 microns, about 15 microns, about 10 microns, about 5 microns, or the like. In a particular embodiment, the average diameter of a plurality of particles of the starch component is about 15 microns. In embodiments where silica is present, the average diameter of the plurality of particles of the silica may be about 25 microns, about 15 microns, about 10 microns, about 9 microns, about 8 microns, about 7 microns, about 6 microns, about 5 microns, about 4 microns, about 3 microns, about 1 microns, or the like, or between about 10 microns and about 1 micron. In some embodiments, the average diameter of a plurality of particles is between about 5 microns and about, 100 microns, or between about 5 microns and about 50 microns, or between 10 microns and 35 microns.

In embodiments where a dry shampoo composition is provided as an aerosol (e.g., an aerosol dry shampoo composition), the composition comprises a propellant and a dry shampoo composition. Suitable propellants will be known to those of ordinary skill in the art. In some embodiments, the propellant may have a lower specific gravity as compared to the dry shampoo composition, thus facilitating propelling the composition from a container (e.g., through a dip tube) as compared to expelling the propellant. Non-limiting examples of propellants include isobutane, liquefied petroleum gas, dimethyl ether, trichlorofluoromethane, dichlorodifluoromethane, dichlorotetrafluorothane, monochlorodifluoromethane, trichlorotrifluoroethane propane, carbon dioxide, nitrous oxide, or combinations thereof. In a particular embodiment, the propellant is isobutane. A propellant may condense to a liquid state in an aerosol container at ambient temperatures. The total percentage weight of the propellant may between about 50% and about 90%, between about 60% and about 85%, between about 65% and about 90%, between about 70% and about 80%, between about 70% and about 75%, between about 80% and about 85%, or between about 75% and about 80%, of the total weight of the aerosol dry shampoo composition (a combination of the dry shampoo composition and the propellant). In a particular embodiment, the propellant is present in an amount between about 75% and about 80%.

An aerosol container may be formed of any suitable material, for example, metal (e.g., aluminum), glass, plastic, or combinations thereof. In most embodiments, the aerosol container is formed essentially of metal. The container may comprise a dip tube and/or a spray nozzle.

In a particular embodiment, a composition of the present invention may comprise a clay material (e.g., stearalkonium hectorite) in an amount (e.g., weight percentage) between about 2% and about 4%, a starch material (e.g., modified starch such as aluminum starch octenylsuccinate) in an amount between about 3% and about 5%, a first carrier material (e.g., alcohol such as ethanol) in an amount between about 1% and about 20%, a second carrier material (e.g., volatile silicone compound such as cyclomethicone) in an amount between about 0.1% and about 15%, and a propellant (e.g., isobutane) in an amount between about 75% and about 80%. In some cases, the composition may further comprise at least one additive (e.g., an oil absorbing additive component such as silica) in an amount between about 0.1% and about 0.5%. The composition may additionally comprise one or more additive components (e.g., perfume, anti-dandruff agent, or cooling agent such as menthol).

In some embodiments, a composition of the present invention consists of or consists essentially of stearalkonium hectorite in an amount (e.g., a weight percentage) between about 2% and about 4%, aluminum starch octenylsuccinate in an amount between about 3% and about 5%, ethanol in an amount between about 1% and about 20%, cyclomethicone in an amount between about 0.1% and about 15%, silica in an amount between about 0.1% and about 0.5%, and isobutane in an amount between about 75% and about 80%.

In some embodiments, an aerosol dry shampoo composition consists or consists essentially of stearalkonium hectorite in a weight percentage between about 2% and about 4%, aluminum starch octenylsuccinate in a weight percentage between about 3% and about 5%, ethanol in a weight percentage between about 1% and about 20%, cyclomethicone in a weight percentage between about 0.1% and about 15%, silica in a weight percentage between about 0.1% and about 0.5%, isobutane in a weight percentage between about 75% and about 80%, and at least one additive in a weight percentage between about 0.1% and about 10%.

In some embodiments a composition of the present invention does not comprise additional components which may leave a colored residue on the hair. For example, the composition may not comprise talc, chalk, or other compounds known to leave a white residue. In some cases, the composition does not comprise magnesium stearate and/or rice protein. In embodiments where a composition comprises a low amount of starch material (e.g., less than about 8%), the composition may comprise an increased amount of clay material (e.g., up to about 10%). An increased amount of a clay material such as stearalkonium hectorite may help to balance and replace some of the oil-removal efficacy that may be lost by a low amount of starch material.

The aerosol dry shampoo composition may be prepared using any suitable technique, as will be known to those of ordinary skill in the art. In some cases, the aerosol dry shampoo composition may be prepared by mixing an appropriate amount of a liquefied gaseous propellant and a dry shampoo composition under pressure, followed by packing the mixture in an aerosol container. In other cases, an aerosol container may be loaded with a dry shampoo composition (e.g., as a powder, slurry, or liquid), followed by pressurizing the container with a propellant and sealing the container.

The dry shampoo compositions of the present invention may be utilized conventionally. An effective amount of the dry shampoo composition may be applied to hair, preferably dry hair. In some cases, the application of the shampoo may encompass massaging or working the shampoo in the hair such that all or most of the hair proximate the scalp is contacted. The term “effective amount,” as used herein, is an amount which is effective in improving the appearance of the hair (e.g., reducing oil content or improving aesthetics). In some cases, the dry shampoo composition may be left in contact with the hair for a period of time after which it may be at least partially removed (e.g., by brushing the hair). The dry shampoo compositions may be useful in settings where a subject does not have access to or may not be exposed to water (e.g., wilderness trips, or hospitals), and/or to preserve and/or add body to a hairstyle without the need for washing the hair.

In some embodiments, a composition of the present invention may be provided as a kit. A “kit” typically defines a package including at least one composition of the invention and instructions. “Instructions” may define a component of promotion, and typically involve written instructions on or associated with packaging of compositions of the invention. Instructions also can include any oral or electronic instructions provided in any manner.

As used herein, a “subject” or a “patient” refers to any mammal, for example, a mammal that has hair or fur. Examples of subjects or patients include a human, a non-human primate, a cow, a horse, a pig, a sheep, a goat, a dog, a cat, or a rodent such as a mouse, a rat, a hamster, or a guinea pig. Generally, the invention is directed toward use with humans.

The above descriptions of applications for the inventive compositions and methods are not intended to be exhaustive, and merely illustrate some of the possible embodiments and uses of this invention.

The function and advantage of these and other embodiments of the present invention may be more fully understood from the example below. The following example, while illustrative of certain embodiments of the invention, does not exemplify the full scope of the invention.

### Example 1

The following is a non-limiting example of an aerosol dry shampoo composition. In this embodiment, the aerosol dry shampoo comprises the following components:

| Component | Range (% by wt.) |
|---|---|
| Alcohol, 200 pf | 1-20 |
| Cyclomethicone | 0.1-15 |
| Aluminum Starch Octenylsuccinate | 3-5 |
| Stearalkonium Hectorite | 2-4 |
| Silica | 0.1-0.5 |
| Isobutane | 75-80 |

### Example 2

The following is a non-limiting example of an aerosol dry shampoo composition comprising a hair-fixative polymer. In this embodiment, the aerosol dry shampoo comprises the following components:

| Component | Range (% by wt.) |
|---|---|
| Alcohol, 200 pf | 1-20 |
| Polyurethane-14 (and) AMP-Acrylates Copolymer | 1-2 |
| Cyclomethicone | 0.1-15 |
| Aluminum Starch Octenylsuccinate | 3-5 |
| Stearalkonium Hectorite | 2-4 |
| Silica | 0.1-0.5 |
| Isobutane | 75-80 |

### Example 3

The following is a non-limiting example of an aerosol dry shampoo composition comprising deodorizing agents. In this embodiment, the aerosol dry shampoo comprises the following components:

| Component | Range (% by wt.) |
|---|---|
| Alcohol, 200 pf | 1-20 |
| Cyclomethicone | 0.1-15 |
| Aluminum Starch Octenylsuccinate | 3-5 |
| Stearalkonium Hectorite | 2-4 |
| Beta-cyclodextrin | 0.1-0.3 |
| Zinc undecylenate | 0.1-0.3 |
| Silica | 0.1-0.5 |
| Isobutane | 75-80 |

### Example 4

The following is a non-limiting example of an aerosol dry shampoo composition comprising natural pediculicides. In this embodiment, the aerosol dry shampoo comprises the following components:

| Component | Range (% by wt.) |
|---|---|
| Alcohol, 200 pf | 1-20 |
| Cyclomethicone | 0.1-15 |
| Aluminum Starch Octenylsuccinate | 3-5 |
| Stearalkonium Hectorite | 2-4 |
| Neem Oil | 1-3 |
| Tea Tree Oil | 1-3 |
| Silica | 0.1-0.5 |
| Isobutane | 75-80 |

While several embodiments of the invention have been described and illustrated herein, those of ordinary skill in the art will readily envision a variety of other means and structures for performing the functions and/or obtaining the results or advantages described herein, and each of such variations, modifications and improvements is deemed to be within the scope of the present invention. More generally, those skilled in the art would readily appreciate that all parameters, dimensions, materials, and configurations described herein are meant to be exemplary and that actual parameters, dimensions, materials, and configurations will depend upon specific applications for which the teachings of the present invention are used. Those skilled in the art will recognize, or be able to ascertain using no more than routine experimentation, many equivalents to the specific embodiments of the invention described herein. It is, therefore, to be understood that the foregoing embodiments are presented by way of example only and that, within the scope of the appended claims and equivalents thereof, the invention may be practiced otherwise than as specifically described. The present invention is directed to each individual feature, system, material and/or method described herein. In addition, any combination of two or more such features, systems, materials and/or methods, provided that such features, systems, materials, and/or methods are not mutually inconsistent, is included within the scope of the present invention.

In the claims (as well as in the specification above), all transitional phrases or phrases of inclusion, such as “comprising,” “including,” “carrying,” “having,” “containing,” “composed of,” “made of,” “formed of,” “involving” and the like shall be interpreted to be open-ended, i.e., to mean “including but not limited to” and, therefore, encompassing the items listed thereafter and equivalents thereof as well as additional items. Only the transitional phrases or phrases “consisting of” and “consisting essentially of” are to be interpreted as closed or semi-closed phrases, respectively. The indefinite articles “a” and “an,” as used herein in the specification and in the claims, unless clearly indicated to the contrary, should be understood to mean “at least one.”

All references cited herein, including patents and published applications, are incorporated herein by reference. In cases where the present specification and a document incorporated by reference and/or referred to herein include conflicting disclosure, and/or inconsistent use of terminology, and/or the incorporated/referenced documents use or define terms differently than they are used or defined in the present specification, the present specification shall control.

What is claimed is:

1. An aerosol dry shampoo composition, comprising:
a carrier material in a weight percentage between about 1% and about 35%;
a starch material in a weight percentage about 3% and about 5%;
a clay material in a weight percentage about 2% and about 4%; and
a propellant in a weight percentage about 75% and about 80%.

2. The composition of claim 1, wherein the carrier material comprises a first type of carrier material and a second type of carrier material.

3. The composition of claim 2, wherein the first type of carrier material is a volatile alcohol and the second type of carrier material is a volatile silicone compound.

4. The composition of claim 3, wherein the volatile alcohol in present in a weight percentage between about 1% and about 20% and the volatile silicone compound is present in a weight percentage between about 0.1% and about 15%.

5. The composition of claim 3, wherein the volatile alcohol is ethanol.

6. The composition of claim 3, wherein the volatile silicone compound is cyclomethicone.

7. The composition of claim 1, wherein the starch is a modified starch

8. The composition of claim 7, wherein the modified starch is aluminum starch octenylsuccinate

9. The composition of claim 1, wherein the clay is a hectorite clay.

10. The composition of claim 9, wherein the clay is stearalkonium hectorite.

11. The composition of claim 1, wherein the propellant is isobutane.

12. The composition of claim 1, further comprising at least one additive component.

13. The composition of claim 12, further comprising at least two additive components.

14. The composition of claim 12, wherein the at least one additive component is silica.

15. The composition of claim 12, wherein the at least one additive component is a perfume.

16. The composition of claim 12, wherein the at least one additive components is present in a weight percentage between about 0.01% and about 10%, or between about 0.1% and about 5%.

17. The composition of claim 16, wherein the at least one additive components is present in a weight percentage between about 0.1% and about 1%.

18. The composition of claim 17, wherein the at least one additive components is present in a weight percentage between about 0.1% and about 0.5%.

19. The composition of claim 1, wherein:
the clay material is stearalkonium hectorite;
the starch material is aluminum starch octenylsuccinate;
the carrier material comprises ethanol in a weight percentage between about 1% and about 20% and cyclomethicone in a weight percentage between about 0.1% and about 15%;
the propellant is isobutane; and
further comprising silica in a weight percentage between about 0.1% and about 0.5%.

20. An aerosol dry shampoo composition, consisting essentially of:
stearalkonium hectorite in a weight percentage between about 2% and about 4%;
aluminum starch octenylsuccinate in a weight percentage between about 3% and about 5%;
ethanol in a weight percentage between about 1% and about 20%;
cyclomethicone in a weight percentage between about 0.1% and about 15%;
silica in a weight percentage between about 0.1% and about 0.5%; and
isobutane in a weight percentage between about 75% and about 80%.

21. (canceled)

22. The composition of claim 20, wherein the composition does not comprise magnesium stearate.

23. The composition of claim 20, wherein the starch is provided as a plurality of particles.

24. The composition of claim 23, wherein the average diameter of the plurality of particles is about 15 microns, or between about 5 microns and about 100 microns, or between about 5 microns and about 50 microns, or between about 10 microns and about 35 microns.

25. The composition of claim 12, wherein the at least one additive component is a hair-fixative polymer, a deodorizing agent, an anti-dandruff agent, a pediculicide, or combinations thereof.

26. The composition of claim 1, wherein the composition further comprises between 0.5% and about 5.0%, or between about 1% and about 3%, or between about 1% and about 2% of a hair-fixative polymer.

27. The composition of claim 1, wherein the composition further comprises between 0.1% and about 5.0%, or between about 0.1% and about 3%, or between about 0.1% and about 1%, or between 0.1 and about 0.5% of a deodorizing agent.

28. The composition of claim 1, wherein the composition further comprises between 0.5% and about 10.0%, or between about 1% and about 8%, or between about 2% and about 6% of a pediculicide.

29. The composition of claim 19, further comprising between about 1% and about 2% of polyurethane-14 (and) AMP-acrylate copolymer.

30. The composition of claim 19, further comprising between about 0.1% and about 0.3% of beta-cyclodextrin and/or between about 0.1% and about 0.3% zinc undecylenate.

31. The composition of claim 19, further comprising between about 1% and about 3% of neem oil and/or between about 1% and about 3% tea tree oil.

32. An aerosol dry shampoo composition, consisting essentially of:
stearalkonium hectorite in a weight percentage between about 2% and about 4%; aluminum starch octenylsuccinate in a weight percentage between about 3% and about 5%;
ethanol in a weight percentage between about 1% and about 20%;
cyclomethicone in a weight percentage between about 0.1% and about 15%;
silica in a weight percentage between about 0.1% and about 0.5%;
isobutane in a weight percentage between about 75% and about 80%; and
at least one additive in a weight percentage between about 0.1% and about 10%.

33. (canceled)

34. (canceled)

35. (canceled)
